(19) United States
(12) Patent Application Publication
(10) Pub. No.: US 2021/0315509 A1
(43) Pub. Date: Oct. 14, 2021

(54) Neural Interface

(71) Applicants: Imperial College of Science, Technology and Medicine, London (GB); University of Maribor, Maribor (SI)

(72) Inventors: Dario FARINA, Birmingham (GB); Deren Yusuf BARSAKCIOGLU, London (GB); Ales HOLOBAR, Zrece (SI)

(21) Appl. No.: 17/270,361

(22) PCT Filed: Aug. 23, 2019
(86) PCT No.: PCT/GB2019/052372

§ 371 Date: Feb. 22, 2021

(30) Foreign Application Priority Data
Aug. 23, 2018 (GB) 1813762.0

(51) Int. Cl. A61B 5/395; A61B 5/397

(52) U.S. Cl. CPC A61B 5/395; A61B 5/397

(57) ABSTRACT

Surface electromyography signals of a nervous system are obtained; a separation matrix is generated based on electromyography signals obtained over a first time period using a training module; one or more motor neuron action potentials for single motor neurones are detected based on said electromyography signals and said separation matrix, wherein said electromyography signals are provided over a second time period shorter than said first time period; and an output is generated in the form of a time-series indicative of motor neuron activity.

### FIELD

The present specification relates to neural interfaces, for example for use as part of a human machine interface (HMI).

### BACKGROUND

Neural interfaces and human-machine interfaces (HMIs) offer significant potential benefits to people who suffer from neuromuscular disorders, paralysis and amputations through advancement of scientific knowledge and development of rehabilitation devices. HMIs can also offer diagnostic tools for the clinical sector. Moreover, with a reliable electrical output signal, HMIs could enable a wide variety of systems to be controlled.

Many neural interface paradigms have been proposed. However, there remains a need for alternative and improved neural interfaces and human-machine interfaces.

### SUMMARY OF THE INVENTION

In an embodiment, there is provided an apparatus (e.g. a human-machine interface) comprising: a neural interface for obtaining surface electromyography signals of a nervous system; a training module (e.g. an adaptive training module) that generates a separation matrix based on first electromyography signals obtained over a first time period; and a decomposition module (e.g. a real-time decomposition module) for detecting (or generating) one or more motor neuron action potentials for single motor neurones based on second electromyography signals and said separation matrix, wherein said second electromyography signals are generated over a second time period shorter than said first time period, and generating an output in the form of a time-series indicative of motor neuron activity. The second time period may be sufficiently short that the process can be carried out on-the-fly.

The training module may comprise a convolutive sphering module in which the first obtained electromyography signals are extended and whitened.

A peak detection module may be provided for generating the output by processing said one or more motor neuron action potentials to provide a time-series indicative of motor neuron that are fired. The peak detection module may form part of the decomposition module.

The training module may further comprise an iteration module for refining the separation matrix.

The neural interface may comprise (or receive signals from) an electrode array for measuring surface electrical signals. A signal conditioning module may be provided for generating the surface electromyography signals from the surface electrical signals. The signal conditioning module may provide amplification, filtering and/or digitisation.

The decomposition module may generate said motor neuron action potentials by matrix multiplication of said electromyography signals and said separation matrix.

The decomposition module may extract discharge timing of motor neurons, enabling decoding of instructions from individual motor neurons to drive specific outputs at specific times.

The training module may implement a blind source separation algorithm.

In some embodiments, the output is provided to an external electrical/electro-mechanical apparatus.

The measured surface electromyography signals may comprise multiple data sources overlapping in time.

In another embodiment, there is provided a method comprising: obtaining (e.g. from, or using, an electrode array) surface electromyography signals of a nervous system; generating a separation matrix based on electromyography signals obtained over a first time period using a training module; detecting (or generating) one or more motor neuron action potentials for single motor neurones based on said electromyography signals and said separation matrix, wherein said electromyography signals are provided over a second time period shorter than said first time period; and generating an output in the form of a time-series indicative of motor neuron activity.

The training module may comprise a convolutive sphering module in which the obtained electromyography signals are extended and whitened.

The method may further comprise generating the output by processing said one or more motor neuron action potentials to provide a time-series indicative of neurons that are fired.

In a further embodiment, there is provided an apparatus as set out above, for use in therapy.

In yet another embodiment, there is provided an apparatus as set out above for use in rehabilitation or assistive devices and/or for use in controlling prosthetics. Other embodiments may include virtual reality gaming and/or working, strength enhancing frames, remote control of digital avatars or robotics etc.

In a further embodiment, the apparatus, as set out above, is used for controlling a system (such as a remote system, a location system, a digital system, a mechanical system or a system incorporating a digital system and a mechanical system).

In another embodiment, there is provided a computer program comprising instructions for causing an apparatus to perform at least the following: obtaining surface electromyography signals of a nervous system; generating a separation matrix based on electromyography signals obtained over a first time period using a training module; detecting (or generating) one or more motor neuron action potentials for single motor neurones based on said electromyography signals and said separation matrix, wherein said electromyography signals are provided over a second time period shorter than said first time period; and generating an output in the form of a time-series indicative of motor neuron activity.

In yet another embodiment, there is provided a computer-readable medium (such as a non-transitory computer readably medium) comprising program instructions stored thereon for performing at least the following: obtaining surface electromyography signals of a nervous system; generating a separation matrix based on electromyography signals obtained over a first time period using a training module; detecting (or generating) one or more motor neuron action potentials for single motor neurones based on said electromyography signals and said separation matrix, wherein said electromyography signals are provided over a second time period shorter than said first time period; and generating an output in the form of a time-series indicative of motor neuron activity.

### BRIEF DESCRIPTION OF THE DRAWINGS

Example embodiments will now be described, by way of example only, with reference to the following schematic drawings, in which:

FIG. 1 is a block diagram of a neural interface;

FIG. 2 is a block diagram of a system in accordance with an example embodiment;

FIG. 3 is a flow chart showing an algorithm in accordance with an example embodiment;

FIG. 4 is a flow chart showing an algorithm in accordance with an example embodiment;

FIG. 5 is a block diagram of a system in accordance with an example embodiment;

FIG. 6 shows example signals in accordance with an example embodiment where the signals are processed by the training module;

FIG. 7 shows the performance of real-time system benchmarked against using synthetic surface EMG;

FIG. 8 shows a block diagram of a system in accordance with an example embodiment;

FIG. 9 shows motor unit discharge patterns for each movement during realtime prosthetic control;

FIG. 10 shows a confusion matrix of a linear support vector machine in accordance with an example embodiment;

FIG. 11 is a block diagram of components of a system in accordance with an example embodiment;

FIG. 12 is a plot showing discharge times in accordance with an example embodiment;

FIG. 13 shows plots of neural and natural force control in accordance with an example embodiment;

FIG. 14 is a plot showing variability in control against the number of motor units in accordance with an example embodiment; and

FIG. 15 shows a block diagram of a system in accordance with an example embodiment.

### DETAILED DESCRIPTION

In the description and drawings, like reference numerals refer to like elements throughout.

Human-machine-interfaces (HMI) present an invaluable prospect for improving, through advancement of scientific knowledge and development of rehabilitation devices, the lives of millions of people who suffer from various neuromuscular disorders, paralysis and amputations worldwide. In addition, HMI have far reaching applications beyond the clinic. With consumer electronics and Internet-of-Things (IoT) devices becoming integral parts of human daily life, HMI also presents the opportunity for humans to control their environment through their thoughts. HMI systems typically include a neural interface which taps into motor and sensory pathways to observe (i.e. record) and modulate (i.e. stimulate) the activity of the nervous system. Robust, reliable, accurate, adaptive, high-information throughput and easy-to-deploy neural interface technologies contribute to realising the potential and fulfilling the promises of future HMI. In this document, the inventors disclose such a device for neural interfaces suitable for a range of applications including research, clinical and consumer electronics use. The device achieves the neural interface through electromyography (EMG) decomposition to monitor the activity of individual motor neurons in the spinal cord.

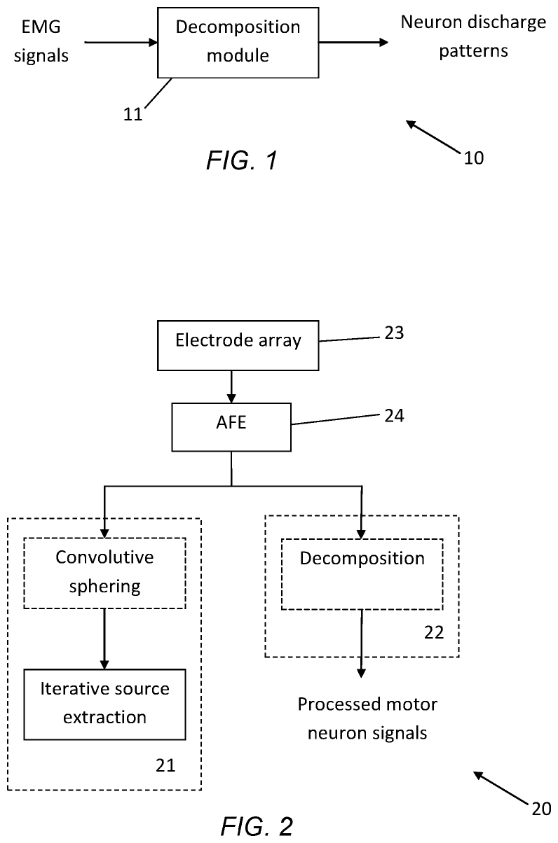

FIG. 1 is a block diagram of a neural interface, indicated generally by the reference numeral 10. The neural interface 10 comprising a decomposition module 11 (e.g. a real-time or near real-time decomposition module). The decomposition module 11 receives electromyography (EMG) signals at an input and provides estimates of neuron discharge patterns at an output.

A number of embodiments described herein refer to “real-time” processing (such as real-time decomposition). In the context of this description, the term “real-time” is intended to cover “near real-time” processing in which a small time delay may occur (e.g. a transmission or processing delay). In such near real-time scenarios, no delay of significance to a user occurs.

FIG. 2 is a block diagram of a system, indicated generally by the reference numeral 20, demonstrating processes carried out by a training module 21 and a decomposition module 22 of the system 20 respectively. An electrode array 23 obtains surface EMG signals which are transmitted to the analogue front-end (AFE) 24 of the device. The AFE 24 includes an analogue-to-digital converter (ADC) and is used to amplify, filter and digitise the surface EMG signals provided by the electrode array 23. The output of the AFE 24 is provided to the training module 21 and the decomposition module 22. As described further below, the output of the AFE 24 undergoes analysis by convolutive sphering and iterative source extraction at the training module 21 and undergoes near-real-time decomposition at the decomposition module, which results in processed motor neuron signals.

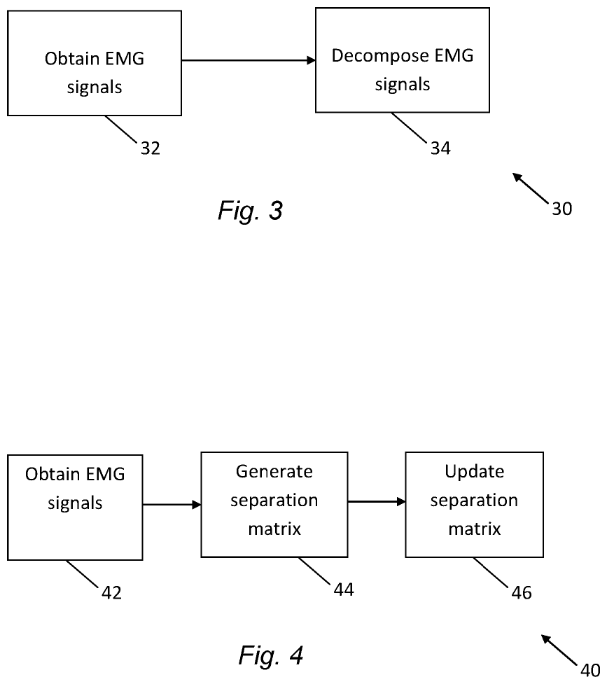

FIG. 3 is a flow chart showing an algorithm, indicated generally by the reference numeral 30, in accordance with an example embodiment. The algorithm 30 starts at step 32 where the EMG signals 32 are obtained (e.g. from the electrode array 23). Next, at step 34, the EMG signals are converted to decomposed EMG signals by the decomposition module 22. The measured surface electromyography signals may comprise multiple data sources overlapping in time.

FIG. 4 is a flow chart showing an algorithm, indicated generally by the reference numeral 40, which forms part of the training module 21. At step 42, EMG signals (which, as noted above, may comprise multiple data sources overlapping in time) are obtained. Next, at step 44, a separation matrix is generated from the obtained EMG signals. At operation 46, the separation matrix is updated.

The algorithms 30 and 40 may be implemented by an HMI (or some other apparatus) comprising: a neural interface for obtaining surface electromyography signals of a nervous system; a training module that generates a separation matrix based on first electromyography signals obtained over a first time period; and a decomposition module for detecting or generating one or more motor neuron action potentials for single motor neurones based on second electromyography signals and said separation matrix, wherein said second electromyography signals are generated over a second time period shorter than said first time period, and generating an output in the form of a time-series indicative of motor neuron activity. The training module may comprise a convolutive sphering module.

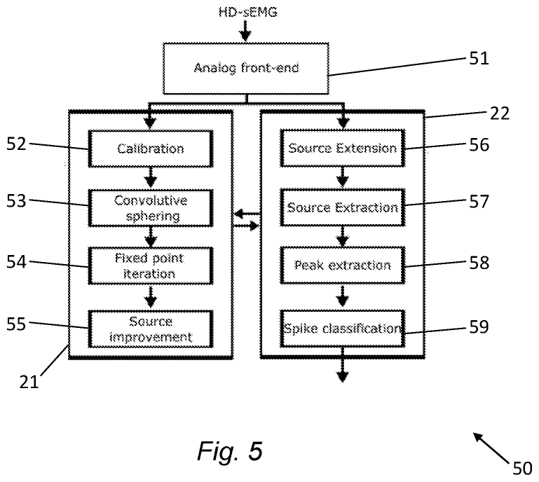

FIG. 5 is a block diagram of a system, indicated generally by the reference numeral 50, showing an analogue front end (AFE) 51 (which may be the same as the AFE 24), the training module 21 and the decomposition module 22. Surface EMG signals (sEMG) are obtained and transmitted to the analogue front-end 51 of a neural interface device. Having been processed and digitised by the AFE, the signals are then transmitted into the training module 21 and into the decomposition module 22.

The training module 21 (e.g. an adaptive training module) employs convolutive blind source separation techniques in order to identify motor units and compute a separation matrix to extract motor unit activity from recorded sEMG signals. The first aspect of the training module comprises a calibration module 52 where average (across all channels) global sEMG amplitude corresponding the user's maximal voluntary contraction is determined, followed by sEMG recordings at constant isometric contraction level. Next, a convolutive sphering module 53 is provided during which the recorded observations are extended (in order to increase the ratio of number of observations to number of sources) and whitened.

Convolutive sphering is described previously by Negro et al. (2016), Multi-channel intramuscular and surface EMG decomposition by convolutive blind source separation, J. Neural Eng., 13(2):026027 (doi: 10.1088/1741-2560/13/2/026027), and comprises two processes; the first process is source extension.

Source extension comprises adding delayed copies of the recorded EMG data from all channels. Considering the EMG as a m×n block of data, wherein m is the number of channels and n is the number of samples recorded for each channel, extension comprises copying the whole data block, delaying it in time, and replicating it under the original data block. This serves the purpose of increasing conditionality of the mixing process by increasing the ratio of the observations (i.e. EMG signal recorded from channels) and sources (i.e. motor neuron discharges). In other words, extending the observations (i.e. EMG recorded from channels) can be used to ensure at least the same number of equations as unknown parameters (i.e. sources) are provided to solve the system of equations describing the mixing process that underlies EMG generation model given in Negro et al. (2016).

The second process involved in convolutive sphering is whitening. Whitening is a mathematical transformation performed on the recorded EMG data to ensure that components (i.e. sources in our case) are uncorrelated to each other. This is a standard pre-processing procedure for many blind source separation algorithm. This step serves the purpose of reducing the parameters that need to be estimated by the blind source separation algorithm.

After the convolutive sphering step, a fixed point iteration module 54 implements a fixed-point iteration algorithm, with a contrast function optimising the sparsity of the extracted independent sources, extracts separation vectors wᵢ for each estimated source sᵢ.

The extracted wᵢ and sᵢ are further refined in a second iterative procedure that estimates the pulse trains with peak detection and K-means classification (signal and noise classes). The second iterative loop calculates a separation vector from estimated discharge timings until reaching a minimum discharge variability, computes Silhouette measure (SIL) and typically accepts the separation vector if the SIL measure is above a threshold (e.g. 0.9).

The training module 21 establishes a separation matrix B containing separation vectors wᵢ, signal (scᵢ) and noise (ncᵢ) cluster centroids for each source, and μ=[μ₁, μ₂, . . . , μₖ]^{T} where k is the number of extended observations and μ is the mean value of each observation during the training recording. These are then used by decomposition module 22 to perform (real-time) decomposition and convert the recorded sEMG signal into a series of motor neuron action potential discharges (i.e. spikes).

Within the decomposition module 22, the EMG signals are first extended using a source extension module 56 (as described further below), then the decomposition module 22 subtracts μ from the extended observations, and then extracts the sources using a source extraction module 57 following multiplication with B. Unlike in the training module 22, the extended sources are not spatially whitened since computation of a whitening matrix involves computationally very expensive singular value decomposition. Instead, B is designed to operate on extended unwhitened observations. This is achieved during the training phase where a whitening matrix W is computed, and the extracted separation vectors are transformed using W.

Following source extraction 57, peaks are detected and extracted using peak extraction module 58 from each squared source vector (sᵢ²), and a distance metric (e.g. Euclidean distance, absolute difference, etc.) between each detected peak and cluster centroids, scᵢ and ncᵢ, are computed. Based on computed distances, spike classification unit 59 decides whether the detected peaks correspond to the discharges of the motor units or noise. Finally, the time occurrence (i.e. timestamp) of each detected motor unit spike is output along with information about which motor unit it belongs to (i.e. spike label).

Thus, the peak detection module may provide a time-series indicative of motor neuron that are fired. The said output may comprise the discharge times as well as the labels (i.e. indicating to which motor neuron the discharge time belongs).

The system 50 may provide an output to an external electrical/electro-mechanical apparatus, such as a prosthetic or an assistive device.

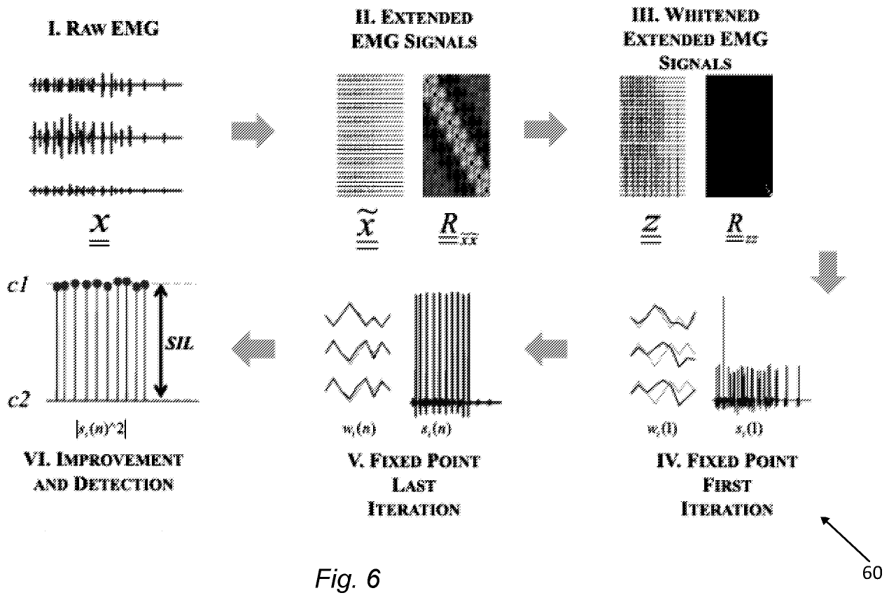

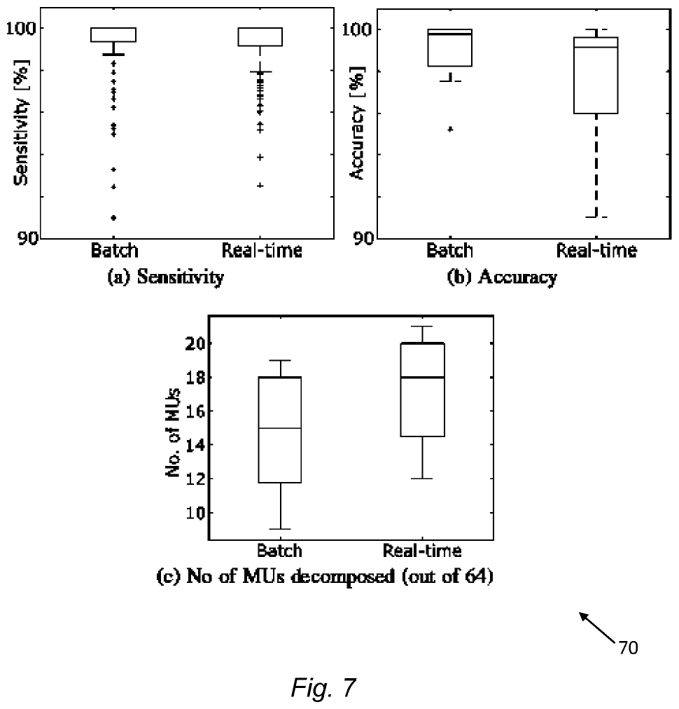

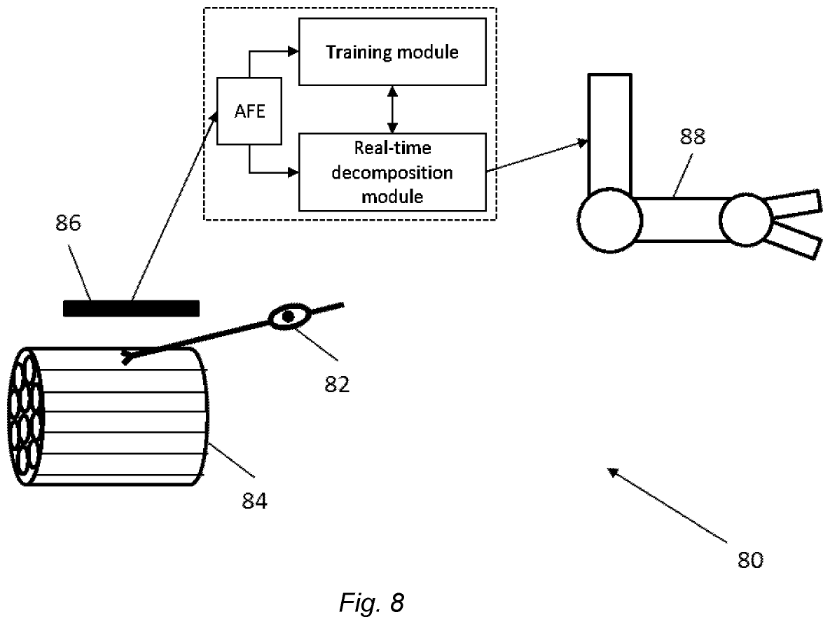

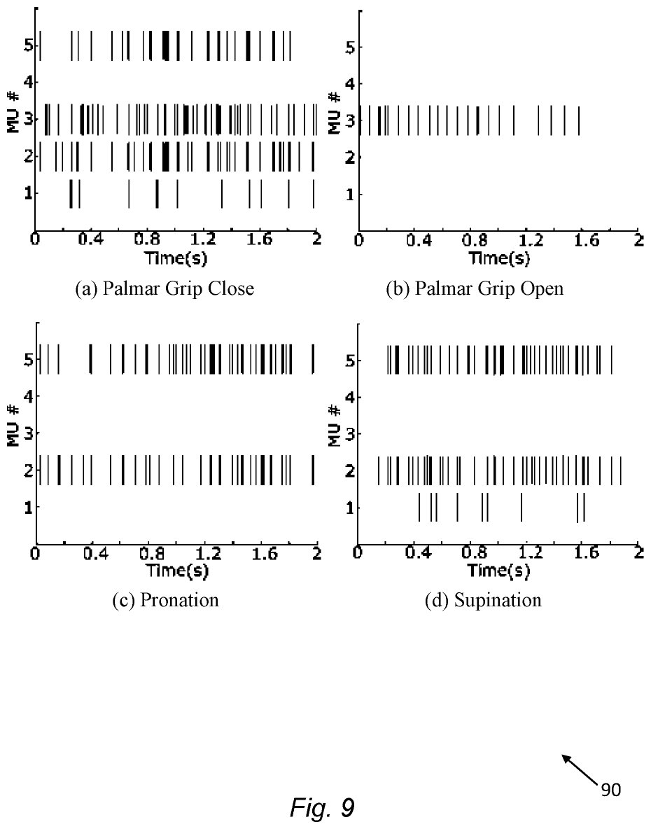

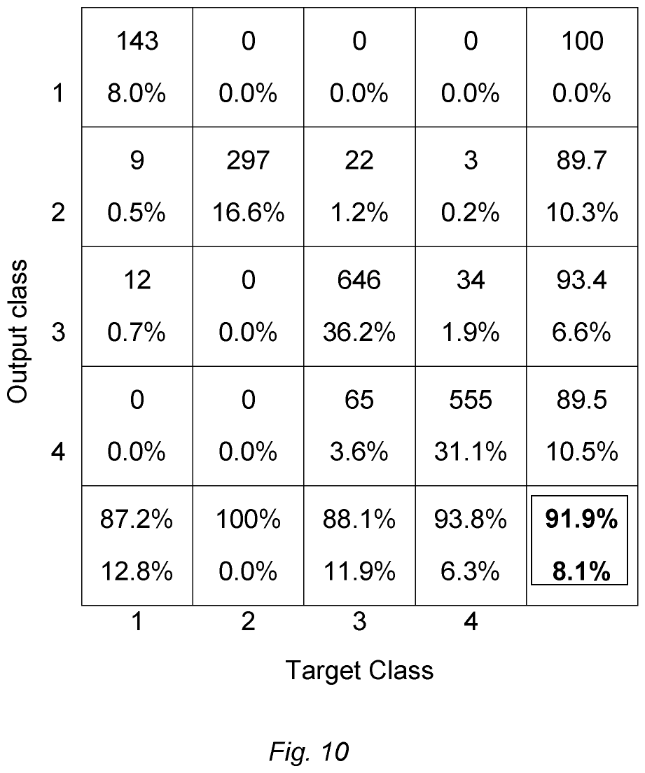

As described further below, FIGS. 6 to 10 show aspects of example uses of the principles described herein. FIG. 6 shows example signals, indicated generally by the reference numeral 60, in accordance with an example embodiment where the signals are processed by the training module. FIG. 7 shows the performance, indicated generally by the reference numeral 70, of a real-time system benchmarked against using synthetic surface EMG. FIG. 8 shows a block diagram, indicated generally by the reference numeral 80, in accordance with an example embodiment. FIG. 9 shows motor unit discharge patterns, indicated generally by the reference numeral 90, for each movement during real-time prosthetic control. FIG. 10 shows a confusion matrix of a linear support vector machine in accordance with an example embodiment.

In one worked example of the training module, shown in FIG. 6, three synthetic sources were mixed in three observations. The synthetic MUAP shapes had a duration of 9 samples. Therefore, for clarity and graphical purposes, FIG. 6 shows the steps of the algorithm with an extension factor equal to 9. However, the performed the calculation using an actual extension factor of 30 (>3 sources×9 samples). According to FIG. 6, Panel (I) shows the raw synthetic EMG mixture. The raw synthetic EMG mixture was then extended, as shown in Panel (II), the extended EMG measurements are shown left, and the corresponding correlation matrix is shown right. Panel (III) shows the whitened extended EMG measurements (left) and corresponding correlation matrix (approximately diagonal) (right). Panels (II) and (III) therefore demonstrate the process of convolutive sphering described herein. Panel (IV) shows a Projection vector (left) and the estimated source (right) after the first step of the fixed point algorithm.

Panel (V) shows the Projection vector (left) and the estimated source (right) after the last step of the fixed point algorithm. Finally, panel (VI) shows improvement of the estimation (see FIG. 3 for details) and calculation of the SIL measure. The SIL is a normalized measure of the distance between the clusters of the detected points (c1) and the cluster of the noise values (c2).

In another worked example, synthetic sEMG datasets were generated. Each dataset simulates sEMG signals recorded over 192-channels, with 64 active motor units (MUs). A total of 6 datasets were generated. Compared to offline decomposition, the system achieved median sensitivity and accuracy within 0.5% for the detected motor units (FIG. 7).

For validation with experimental sEMG recordings, the system was connected to a 64-channel high-density electrode matrix (OT Bioelletronica ELSCH064NM2) and measurements were made from the Tibialis Anterior (TA) and Flexor Digitorum Superficialis (FDS) muscles of two subjects. The sEMG signals were bandpass filtered (10-500 Hz), sampled at 2048 Hz, and A/D converted to 16 bits by the AFE. During recordings, both the spiking activity of MUs (decomposed in real-time) and raw sEMG recordings (for post-recording validation) were recorded simultaneously. The Rate-of-Agreement (RoA) measure was used to quantify the performance of realtime decomposition. RoA (%) is defined by:

$$RoA [ \% ] = \frac{{c}_{j}}{{c}_{j} + {RT}_{j} + {B}_{j}} \times 100 \%$$

where cⱼ is the total number of discharges of the jth motor unit identified by both real-time and offline (batch) decomposition algorithms, RTⱼ is the number of discharges identified by the real-time system only, and Bⱼ is the number of discharges identified by offline batch processing algorithm only (using the raw sEMG data recorded during experiments). If cⱼ was more than 30%, two motor unit discharge patterns were considered to be generated by the same motor unit. The results (Table I) reveal an average RoA of 83% across all muscles and contraction levels recorded.

TABLE I
Decomposition accuracy of the real-
time system for 64-channel HD-sEMG
recordings across various muscles and
contraction levels [% MVC].

| % MVC | RoA % |
|---|---|
| TA 10 | 92.3 ± 4.2 |
| | (85.9-98.8) |
| TA 20 | 78.0 ± 16.0 |
| | (55.8-97.1) |
| TA30 | 80.0 ± 22.4 |
| | (31.1-95.8) |
| TA 10 | 85.1 ± 9.3 |
| | (64.3-93.6) |
| TA 20 | 97.3 ± 1.0 |
| | (95.9-99.0) |
| TA 30 | 90.4 ± 5.3 |
| | (84.7-96.5) |
| FDS 10 | 82.5 ± 16.6 |
| | (60.4-98.2) |
| FDS 20 | 82.9 ± 9.3 |
| | (70.7-96.4) |
| FDS 30 | 57.2 ± 23.3 |
| | (35.5-81.7) |

TA: Tibialis Anterior; FDS: Flexor digitorum superficialis; RoA: Rate of Agreement (mean ± SD (min-max))

In one theoretical example, as shown in FIG. 8, the resulting sEMG signal, of a motor neuron 82 signalling to a muscle fibre 84 in the limb of a subject, is obtained by an electrode 86. The obtained sEMG signal is transmitted to the AFE and then transmitted to and processed by both the training module and the decomposition module. The data obtained after processing is then used to send an electronic instruction to a prosthetic limb 88 thereby resulting in the control of the limb by the subject's nervous system.

In a worked example, the system described herein was used in a prosthetic hand control (Michelangelo Hand, Ottobock) paradigm where the movements along 2-DoF (hand open/close and hand pronate/supinate) were controlled in real-time by decomposed MU activity. The control was achieved through a support vector machine (SVM) framework, where the input to the SVM classifier were the filtered discharge rates of each motor unit.

A graphical user interface (GUI), that displays decomposed motor units or average global sEMG as feedback in real-time, was also developed to facilitate the experiments. The discharge rate was calculated over intervals of 128 samples (62.5 ms) by summing the spikes detected for each MU in the window. These values were continuously fed into a second order Butterworth filter with low pass cut-off of 100 Hz.

During the training stage for the real-time system, the subject was asked to follow up a force ramp at %10, %20 and %30 contraction levels (45 seconds each) in order to extract MUs. A total of 5 MUs were decomposed from the subject. During the training for the SVM classifier, the subject was asked to perform isometric contractions of various hand movements while the discharges of all MUs were displayed as feedback via computer screen.

SVM classifier was trained on identified distinct MU activity patterns which were mapped into four different hand/wrist movements: (1) Palmar grip close, (2) Palmar grip open, (3) Pronation, and (4) Supination (see FIG. 9). FIG. 10 presents the confusion matrix for classifying movements across 2-DoF, indicating a classification accuracy of 91.9%.

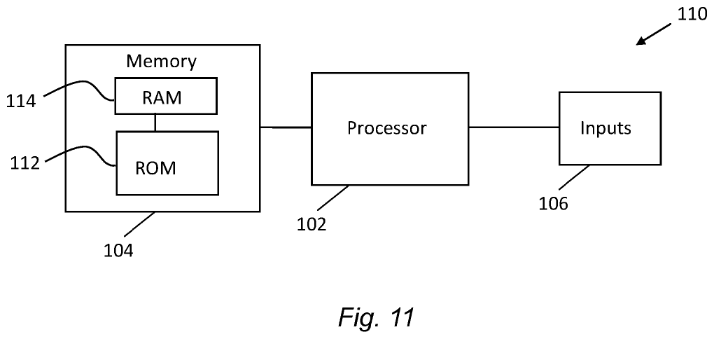

FIG. 11 is a block diagram, indicated generally by the reference numeral 110, of components of a system in accordance with an example embodiment. The system 110 comprises a processor 102, a memory 104 and one or more inputs 106. The memory comprises a ROM 112 and a RAM 114. The processor is connected to each of the other components in order to control the operation thereof.

The ROM 112 of the memory 114 may store an operating system and software applications. The RAM 114 may be used by the processor 102 for the temporary storage of data. The operation system may contain code which, when executed by the processor, implements aspects of the algorithms 30 or 40 described above.

Results

Systems in accordance with the principles described herein have been tested and a number of results obtains, as outlined below.

Accuracy of Online Decomposition

One participant (male, 28 years, 181 cm height) tested online control of motor neurons innervating the tibialis anterior, first dorsal interosseus, abductor digiti minimi, and extensor digitorum muscles, as representative muscles of the lower and upper limb.

The tests were performed by providing visual feedback to the participant and requesting him to progressively recruit 8 to 10 motor neurons, to maintain the activity of the recruited neurons for approximately 10 seconds, and then to progressively derecruit. EMG signals recorded from grid electrodes were online decomposed and the spiking activity of the decoded neurons was shown as visual feedback to the participant. The result of the online decomposition was also stored and compared with the subsequent offline, partly manual decomposition. The results of the accuracy analysis are reported as rate of agreement between online and offline decomposition for all muscles investigated (see the “Methods” discussion below).

The online decomposition identified 5 to 12 motor units, depending on the muscle investigated. Table II reports the rate of agreement between online and offline (partly manual) decomposition as well as the discharge rate and discharge variability of the identified motor units. The values of agreement are >90% for all muscles and are similar across muscles. The values for discharge rate and discharge variability are in agreement with known physiological values.

These results indicate high accuracy of the online identification of motor neuron spiking and generalisability of the approach to muscles with different architecture and control properties.

TABLE II
Decomposition accuracy of the real-time system measured with
64-channel HD-sEMG recordings across various muscles.

| | | | Average | Average |
|---|---|---|---|---|
| | | Rate of | Discharge | Discharge |
| | # of | Agreement | Rate | variability |
| Muscle | MUs | (RoA) % | (pps) | (CV %) |
| TA | 12 | 99.9 ± 0.2 | 11.7 ± 1.3 | 9.9 ± 1.5 |
| | | (99.4-100) | (10.2-14.0) | (8.1-13.3) |
| FDI | 5 | 99.3 ± 0.6 | 9.5 ± 1.3 | 12.4 ± 2.1 |
| | | (98.3-100) | (7.9-11.2) | (10.3-15.9) |
| ADM | 6 | 95.5 ± 3.1 | 15.6 ± 5.5 | 18.3 ± 4.9 |
| | | (93.2-99.1) | (9.1-22.4) | (13.1-25.4) |
| ED | 7 | 91.6 ± 3.8 | 19.6 ± 5.8 | 21.9 ± 5.5 |
| | | (87.4-94.8) | (12.8-30.8) | (14.4-28.1) |

TA: Tibialis Anterior; FDI: First dorsal interosseus; ADM: Abductor digiti minimi; ED: Extensor Digitorum. Rate of Agreement, discharge rate, and discharge variability are given as mean ± standard deviation (minimum RoA-maximum RoA), across all identified motor units.

The accuracy was further tested in additional ten participants (four female; age: 27.2±5.2 yrs) on the tibialis anterior muscle only. In these extensive tests, the average rate of agreement was 91.2±8.4 (65.6-100)%.

Accuracy in Voluntary Control of Motor Neuron Output

Finally, the ability to control recruitment of motor neurons when providing visual feedback on their activity was investigated and compared the variability in motor neuron control with respect to natural force control. Since force is determined by the low-pass filtering of motor neuron output, it was hypothesized that it is possible to achieve similar accuracy in motor neuron output control as in force control with a visual feedback. A total of ten participants (four female; age: 27.2±5.2 yrs) were recruited for this experiment and HD-sEMG were recorded from the tibialis anterior muscle during ankle dorsiflexion.

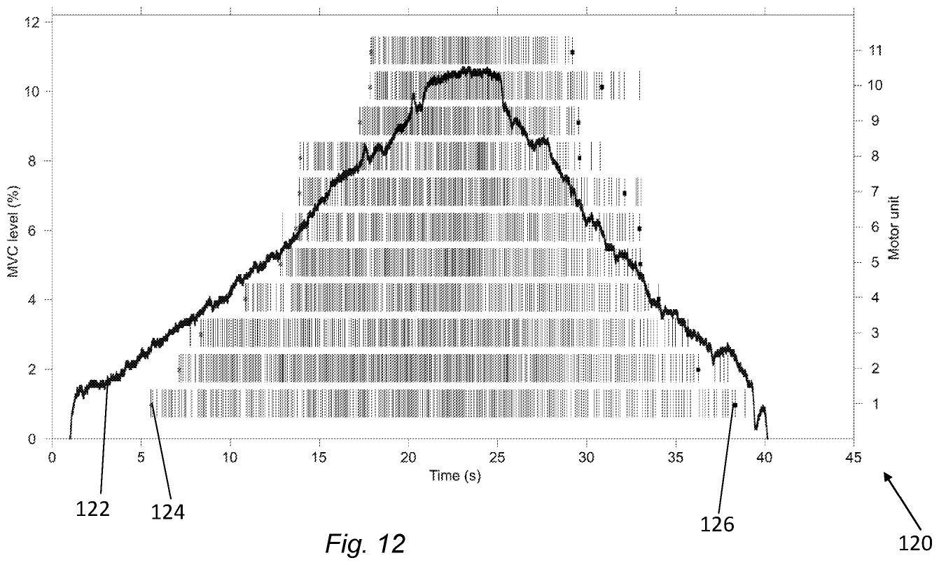

In the first part of the experiment, real-time feedback on motor unit activities was provided to the participants who were asked to progressively recruit one motor unit at a time until reaching 10% MVC force and then derecruit the motor units progressively. FIG. 12 is a plot, indicated generally by the reference numeral 120, showing discharge times during recruitment of decruitment for one participant in accordance with an example embodiment. In the plot 120, motor units are ranked by their recruitment threshold in ascending order from bottom to top. A line 122 indicates the force level (left vertical axis). First dots on the left (such as the dot 124) indicate the recruitment time for each motor unit, while second dots on the right (such as the dot 126) indicate the derecruitment time.

In this example, the participants were instructed to separate recruitment and derecruitment of subsequent units by a few seconds (>1 second) in order to prove voluntary control over progressive recruitment/derecruitment. All participants were able to control recruitment and derecruitment of motor units individually. The median time interval (±interquartile range) between recruitment of subsequent motor units was 3.0±3.6 s, while for derecruitment it was 2.8±7.1 s. This result confirms that all participants were able to recruit/derecruit single motor units when provided visual feedback on discharge patterns of individual motor units.

In the second part of the experiment, the participants were presented two modes of visual feedback. The neural feedback was the FCST of the motor units decomposed in real-time. With this feedback, the participants were asked to follow, as closely as possible, a series of targets at varying contraction levels: 2%, 4%, 6%, 8% and 10% of the maximal activation. These targets were repeated twice for each visual feedback type. The force feedback was based on force at the same relative levels used for the neural feedback.

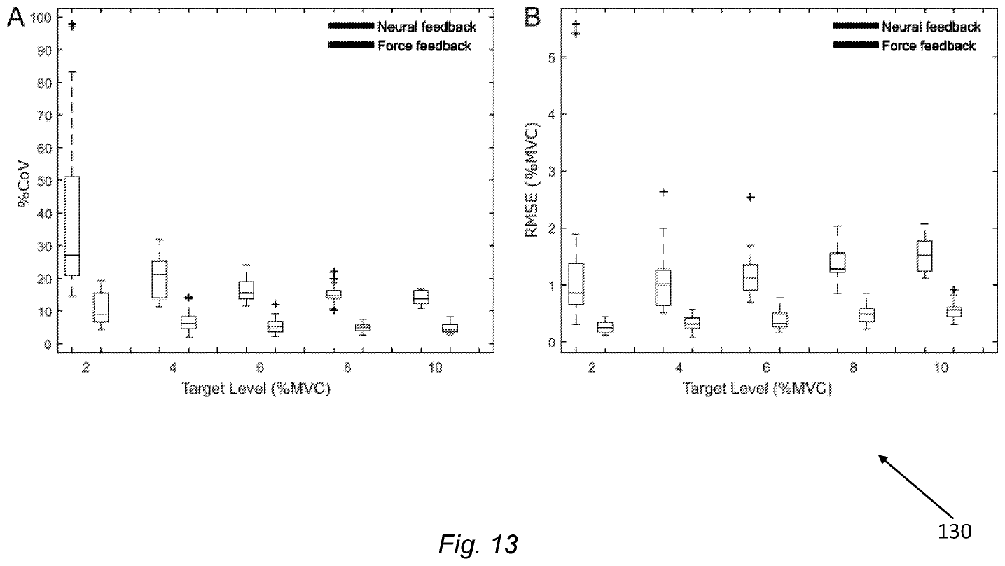

FIG. 13 shows plots, indicated generally by the reference numeral 130, of neural and natural force control. Accuracy in control using motor neuron output and force feedback quantified in percentage coefficient of variation (A) and root mean square error (B).

On average, 11.5±2.1 motor units were decomposed per participant (minimum 8 motor units and maximum 15 motor units). Force feedback had lower overall CoV (median variability of 5.6%) and RMSE (median error of 0.4% MVC) across participants compared to the variability resulting from neural feedback (median variability of 16.0% and RMSE of 1.2% MVC). The largest variability for neural feedback was observed, as expected, for lower contraction levels and variability progressively decreased with contraction level. The difference in variability between force and neural feedback progressively decreased with contraction level and it reached a minimum at 10% contraction (force: 4.1%; neural feedback: 13.7%). This was due to the increasing number of motor units identified when increasing contraction level (2.2±1.8 at 2% MVC; 5.4±2.3 at 4% MVC; 7.0±2.1 at 6% MVC; 9.2±1.8 at 8% MVC; and 10.7±1.7 at 10% MVC). Accordingly, over the entire participant samples and contraction levels, the variability in control was inversely proportional to the number of identified motor units. The variability of both force and neural control decrease with the number of identified motor units. For force control, this was due to the association between the force level and number of identified units (more identified units at greater forces). For neural control, the number of units determined the stability of the cumulative spike train used for control.

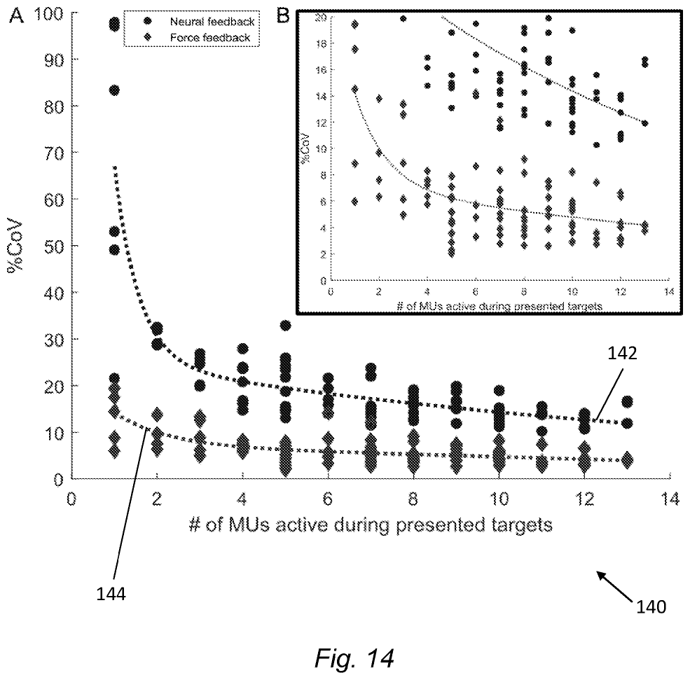

FIG. 14 is a plot, indicated generally by the reference numeral 140, showing variability in control against the number of motor units under neutral control (indicated by circles and the line 142) and under force control (indicated by diamonds and the line 144). The number of active (i.e. discharging) motor units was identified for each presented target across all force levels, trials, and participants. Exponential trend fits of variability values for neural control (the line 142) and force control (the line 144) are also presented (A). A magnified version of A showing that force variability reaches a minimum asymptotic value while variability of neural control approaches this value as the number of motor units involved in the control task increases (B).

When the fitted exponential trends for neural and force control of the plot 140 are extrapolated, it was observed that the neural control and force control has the same variability in control (i.e. two curves intersect) at 73 motor units; while the difference in variability is less than 1% above 40 motor units.

Methods

For all experiments, high-density surface electromyogram (HDsEMG) signals were recorded using 64-electrode adhesive grids (5 columns and 13 rows; gold coated; OT Bioelettronica, Torino, Italy) mounted over the belly of the target muscle. For tests on the tibialis anterior and extensor digitorum muscles, a high-density electrode array with an 8-mm inter-electrode distance was used; while for the first dorsal interosseus and abductor digiti minimi a smaller sized high-density electrode array with an inter-electrode distance of 4-mm was applied. A conductive paste was used to improve the electrode-skin contact. Before the placement of the electrode array, the skin above the target muscle was shaved and cleansed with abrasive paste and ethanol. The signals were recorded in monopolar derivation with a Quattrocento Amplifier (OT Bioelettronica, Torino, Italy), sampled at 2048 Hz, A/D converted to 16 bits, and band passed filtered (10-500 Hz). The force was measured through CCT TF-022 force transducer and recorded, amplified (OT Bioelettronica, Torino, Italy), and bandpass filtered (0-30 Hz).

Together with the real-time decomposition system, a custom designed graphical user interface (GUI) was used during the experiments. The GUI provided feedback on either force, spiking activity of individual motor units, or cumulative spike trains of sets of motor units. For all experiments, participants started with performing the maximum voluntary contraction over a 40-second interval during which they were asked to exert maximum dorsiflexion, abduction or extension depending on the recorded muscle. The maximal contraction was followed by a ramp contraction task that comprised a 4-second ramp trajectory at 2.5% MVC/s, a 10% MVC sustained contraction over 41 seconds. HD-sEMG signals recorded during this contraction were used as the training data for the system to identify motor units and real-time decomposition system parameters. The participants were then asked to slowly recruit one motor unit after another by increasing the contraction level continuously from 0% to 10% MVC while single motor unit spiking of the entire pool was visually presented. According to the estimated recruitment timing and current contraction level, the decomposed motor units were arranged in ascending order based on their recruitment threshold from low to high. Following this initial preparation and calibration phase, the experiments (detailed below) were carried out.

All experimental procedures described in this work were approved by Joint Research Compliance Office under the Imperial College Research Ethics Committee process (reference 18IC4685). All participants gave informed consent according to procedures approved by the ethics committee at Imperial College London.

Experimental Accuracy of Online Decomposition

One healthy participant (male, 28) was recruited for this experiment in order to validate the online decoding of motor unit activity across several muscles. High-density surface electromyogram (HDsEMG) from the tibialis anterior, first dorsal interosseus, abductor digiti minimi, extensor digitorum were recorded in separate experimental sessions, performed in different days. For the tibialis anterior muscle, the foot was fixed into position using an ankle ergometer (NEG1, OT Bioelettronica), with the ankle flexed at 0°, to allow isometric dorsiflexion of the ankle. For the measurements on the upper limb, i.e. abduction of the index finger (first dorsal interosseus), abduction of the little finger (abductor digiti minimi), and extension of the medial four digits of the hand (extensor digitorum), a custom designed experimental setup was used to fix the forearm, the wrist and the hand. It consisted of an adjustable-height platform to ensure the forearm was bent at the elbow at 90° and the hand was pronated at 90°. The force sensor platform was adjusted accordingly and fixed ensuring the digits of the hand relevant to the measurement stayed in contact with the force platform at rest. During the experiment, visual feedback on the discharge behaviour of each identified motor unit and the current contraction level was provided to the participants. Participants were then asked to recruit all identified motor units and to sustain a constant firing rate for 45 s at a contraction force of approximately 10% MVC. The MVC % force level, in this case, was also displayed through the graphical user interface. For the offline analysis, the HDsEMG signals were processed with an offline decomposition algorithm to assess the overall quality of the online decomposition. The offline decomposition was performed with a threshold of SIL>0.9, while the fixed-point algorithm was iterated over 50 times. The results of the offline decomposition were manually edited for maximizing the accuracy. To quantify the performance of real-time decomposition to the offline benchmark, the rate-of-agreement (RoA) was used as a metric. RoA is defined as:

$$RoA \% = \frac{{c}_{j}}{{c}_{j} + R {T}_{j} + {B}_{j}} \times 100 \%$$

where cⱼ is the total number of discharges of the j^{th} motor unit identified by both real-time and offline decomposition algorithms, RTⱼ is the number of discharges identified by the real-time system only, and Bⱼ is the number of discharges identified by offline decomposition only. If cⱼ was more than 30%, the two motor unit discharge patterns were considered to be generated by the same motor unit.

Accuracy in Voluntary Control of Motor Neuron Output

Experiments were performed on ten healthy participants (4 female; age: 27.2±5.2 yrs). Recordings were performed with high-density surface electromyogram (HDsEMG) systems from the tibialis anterior muscle. The foot was locked into position to allow dorsiflexion of the ankle only. During the initial part of the experiment, following initial system calibration, the participants were shown the discharge times of each identified motor unit, decoded in real time from the tibilais anterior muscle. They were then asked to control the discharge behaviour of every motor unit by performing dorsiflexion of the foot in order to recruit one motor unit after another, maintain all motor units discharging at 10% MVC, and de-recruit one motor unit after another. During the offline analysis, motor units were automatically ranked by their recruitment threshold. A motor unit was classified as activated when it discharged action potentials at a firing rate above 4 pps for at least 2 seconds. Eventually, all motor units were ranked in ascending order based on the corresponding force level at the time of activation (recruitment threshold). A similar procedure was used to determine the deactivation. The time point at which the firing rate of a motor unit dropped below 4 pps for at least 2 s was classified as motor unit derecruitment (see FIG. 13).

During the second part of the experiment, participants were provided a series of targets and asked to follow the targets with two types of visual feedback: cumulative spike train and force. All targets were repeated twice for each visual feedback type and all visual feedback signals were filtered with a 4^{th} order Butterworth lowpass filter with a cut-off frequency of 5 Hz.

For each type of visual feedback, the targets consisted of a 4-second ramp trajectory (increasing), followed by a constant contraction level for 32 seconds, and ending with a 4-second ramp trajectory (decreasing). The constant contraction levels were 2%, 4%, 6%, 8% and 10% MVC. The MVC level when using the neural feedback was determined during the initial calibration phase where participants were asked to follow targets at the mentioned force levels, while provided force feedback. The average discharge rates of filtered cumulative spike train were computed for each force level (NScale_{force level}) during the calibration phase. These were then used as the scaling factors during the neural feedback tasks to normalise FCST and convert the filtered cumulative spike train into MVC %.

$${FCST}_{\% MVC} = \frac{F C S T}{N S c a l {e}_{{ }^{force level}}} \times MVC {\%}_{t a r g e t}$$

The force level (as % MVC)—measured through the force sensor—was constantly monitored at all times by the experimenter during all tasks.

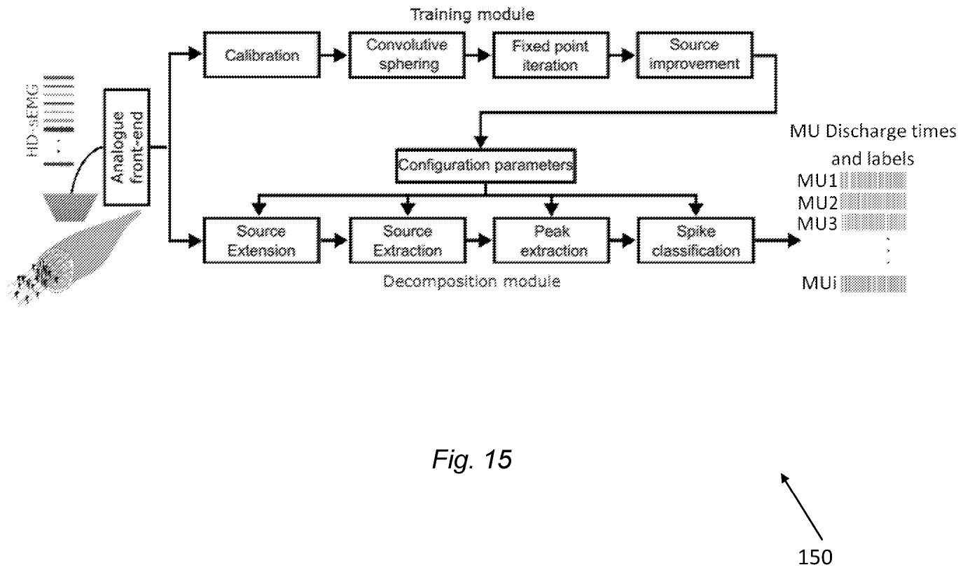

FIG. 15 is a block diagram of a system, indicated generally by the reference numeral 150, showing an analogue front end (AFE) and a decomposition module 22 used in example embodiments.

Embodiments of the present invention may be implemented in software, hardware, application logic or a combination of software, hardware and application logic. The software, application logic and/or hardware may reside on memory, or any computer media.

If desired, the different functions discussed herein may be performed in a different order and/or concurrently with each other. Furthermore, if desired, one or more of the above-described functions may be optional or may be combined.

It will be appreciated that the above described example embodiments are purely illustrative and are not limiting on the scope of the invention. Other variations and modifications will be apparent to persons skilled in the art upon reading the present specification.

Moreover, the disclosure of the present application should be understood to include any novel features or any novel combination of features either explicitly or implicitly disclosed herein or any generalization thereof and during the prosecution of the present application or of any application derived therefrom, new claims may be formulated to cover any such features and/or combination of such features.

Although various aspects of the invention are set out in the independent claims, other aspects of the invention comprise other combinations of features from the described example embodiments and/or the dependent claims with the features of the independent claims, and not solely the combinations explicitly set out in the claims.

1. An apparatus comprising:
a neural interface for obtaining surface electromyography signals of a nervous system;
a training module that generates a separation matrix based on first electromyography signals obtained over a first time period; and
a decomposition module for detecting one or more motor neuron action potentials for single motor neurones based on second electromyography signals and said separation matrix, wherein said second electromyography signals are generated over a second time period shorter than said first time period, and generating an output in the form of a time-series indicative of motor neuron activity.

2. The apparatus as claimed in claim 1, wherein the training module comprises a convolutive sphering module in which the first obtained electromyography signals are extended and whitened.

3. The apparatus as claimed in claim 1, wherein the decomposition module further comprises a peak detection module for generating the output by processing said one or more motor neuron action potentials to provide a time-series indicative of motor neurons that are fired.

4. The apparatus as claimed in claim 1, wherein the training module further comprises an iteration module for refining the separation matrix.

5. The apparatus as claimed in claim 1, wherein said neural interface comprises:
an electrode array for measuring surface electrical signals; and
a signal conditioning module for generating the surface electromyography signals from the surface electrical signals.

6. The apparatus as claimed in claim 1, wherein the training module is an adaptive training module.

7. The apparatus as claimed in claim 1, wherein the decomposition module detects said motor neuron action potentials by matrix multiplication of said electromyography signals and said separation matrix.

8. The apparatus as claimed in claim 1, wherein the decomposition module extracts discharge timing of motor neurons, enabling decoding of instructions from individual motor neurons to drive specific outputs at specific times.

9. The apparatus as claimed in claim 1, wherein the training module implements a blind source separation algorithm.

10. The apparatus as claimed in claim 1, wherein the output is provided to an external electrical/electro-mechanical apparatus.

11. The apparatus as claimed in claim 1, wherein the apparatus is a human-machine interface.

12. A method comprising:
obtaining surface electromyography signals of a nervous system;
generating a separation matrix based on electromyography signals obtained over a first time period using a training module;
detecting one or more motor neuron action potentials for single motor neurones based on said electromyography signals and said separation matrix, wherein said electromyography signals are provided over a second time period shorter than said first time period; and
generating an output in the form of a time-series indicative of motor neuron activity.

13. The method as claimed in claim 12, wherein the training module comprises a convolutive sphering module in which the obtained electromyography signals are extended and whitened.

14. The method as claimed in claim 12, further comprising generating the output by processing said one or more motor neuron action potentials to provide a time-series indicative of neurons that are fired.

15. The apparatus of claim 1, for use in therapy.

16. The apparatus of claim 1, for use in rehabilitation or assistive devices.

17. The apparatus of claim 1, for use in controlling prosthetics.
